(19) United States
(12) Patent Application Publication
(10) Pub. No.: US 2007/0298087 A1
(43) Pub. Date: Dec. 27, 2007

(54) Two-phase mucoadhesive composition

(76) Inventor: James E. Biegajski, Petaluma, CA (US)

Correspondence Address: HOWARD EISENBERG, ESQ., 1220 LIMBERLOST LANE, GLADWYNE, PA, 19035, US

(21) Appl. No.: 11/654,921

(22) Filed: Jan. 18, 2007

Related U.S. Application Data
(60) Provisional application No. 60/816,673, filed on Jun. 27, 2006.

(51) Int. Cl. A61K 9/70

(52) U.S. Cl. 424/443; 424/449

(57) ABSTRACT

A novel two-phase, film-forming, mucoadhesive gel composition has been developed. The composition contains an anhydrous non-aqueous solvent, a water-insoluble polymer that is dissolved in the non-aqueous solvent, and a water-soluble polymer that is dispersed in the non-aqueous solvent. The composition adheres to water-bearing surfaces, such as to moistened skin or mucosal surfaces, and can be used to provide extended, local delivery of pharmaceutically active agents from the composition to skin or a mucosal surface. The composition may be solvent-cast to form a mucoadhesive film, which may contain a polymeric backing layer. The mucoadhesive film may further contain a pharmaceutically active agent and may be used to administer the pharmaceutically active agent to skin or mucosal surfaces.

This application claims the benefit of U.S. Provisional Patent Application No. 60/816,673, filed on Jun. 27, 2006.

### FIELD OF THE INVENTION

This invention relates to the field of mucoadhesives and mucoadhesive films for attachment to a water-bearing surface, such as moistened skin or a mucosal surface. The invention further relates to the fields of providing barrier protection to a surface, such as skin or a mucous membrane, and delivering pharmaceutical agents to the surface over an extended period of time.

### BRIEF DESCRIPTION OF THE FIGURES

FIG. 1 is a diagrammatic representation of a two-phase composition of the invention.

FIG. 2 is a diagrammatic representation of a two-phase composition of the invention situated on buccal mucosa.

FIG. 3 is a diagrammatic representation of the two-phase composition of FIG. 2 following influx of saliva into the composition.

FIG. 4 is a diagrammatic representation of a mucoadhesive film laminate of the invention including a slowly dissolving backing layer.

### DESCRIPTION OF THE INVENTION

It has been discovered that a mucoadhesive, film-forming composition may be prepared by dispersing finely divided particles of a water-soluble polymer, such as poly(ethylene oxide), in an anhydrous solution, such as an alcoholic solution, for example an ethanolic solution, of a non-water soluble polymer such as ethylcellulose. The composition was found to form a cohesive film in situ when topically applied onto a water-bearing surface, such as moistened skin or a mucosal surface like that in the oral cavity, and which film slowly erodes or dissolves due to exposure of the film to the water from the surface, or water from the adjacent body cavity, such as saliva. One way that this film may be used is to provide barrier protection, such as by providing a sealing cover over a skin lesion such as a cut or an oral lesion such as an aphthous ulcer. Another way that the composition may be used is to include in the composition a pharmaceutically active agent. During the extended residence time of the film on the surface, the pharmaceutically active agent present in the film is slowly delivered to the adjacent skin or mucosal surface.

It has also been discovered that the composition of the invention may be cast and dried, such as on a release liner, to produce a dry film. The dry film obtained in this manner may be adhered onto a water-bearing surface, such as moistened skin or a mucosal surface, and, if a pharmaceutically active agent is included within the film, will provide for extended, local delivery of the active agent contained therein. Additionally, a slowly-dissolving backing layer may be situated on top of this mucoadhesive film to further extend its residence time and, if a pharmaceutically active agent is present, thereby extend the delivery of the pharmaceutically active agent.

In one embodiment, the invention is a mucoadhesive composition having two phases. One phase is an anhydrous solvent in which is dissolved a water-insoluble polymer. The anhydrous solvent is preferably volatile. The second phase is a water-soluble mucoadhesive polymer that is insoluble in the anhydrous solvent and is dispersed in the anhydrous solvent. The composition may be applied to a water-bearing surface, such as to moistened skin or to a mucosal surface, and the composition will adhere to the surface. In doing so, the composition will form a film in situ upon the surface. In a preferred embodiment, the mucoadhesive composition is a gel formulation.

In another embodiment, the invention is a composition in the form of a mucoadhesive film. The film is made by thinly applying an amount of the mucoadhesive composition described above to a surface and permitting the anhydrous solvent to evaporate, thereby obtaining the film, a process referred to as “solvent-casting.” The film adheres to a water-bearing surface, such as to moistened skin or to a mucosal surface.

In another embodiment, the invention is a composition in the form of a mucoadhesive film laminate. The laminate contains two layers. The first layer is the mucoadhesive film described in the previous paragraph. The second layer is a backing layer that adheres to the mucoadhesive film. The backing layer may be a polymer film that dissolves slowly in an aqueous medium. Preferably, the backing layer polymer film is made by solvent-casting upon the mucoadhesive film layer.

In another embodiment, the invention is a mucoadhesive composition or film as described above following application and adherence to a water-bearing surface for a time sufficient for the water to enter the composition or film, wherein the mucoadhesive polymer is swollen due to the water and the water-insoluble polymer has precipitated, forming a cohesive film or membrane.

In another embodiment, the invention is any of the compositions described above which further contains a pharmacologic agent. The pharmacologic agent may be dissolved or dispersed in the anhydrous solvent or may be dispersed within the mucoadhesive film.

In another embodiment, the invention is a method for making a mucoadhesive composition. According to this embodiment of the invention, a water-insoluble polymer is dissolved in an anhydrous solvent to form a solution and a water-soluble polymer that is insoluble in the anhydrous solvent is added to the solution and mixed until dispersed. If desired, a polymer that is soluble in water and in the anhydrous solvent may be added by mixing into the dispersion. The water and anhydrous solvent soluble polymer may be used to increase the viscosity of the composition. If desired, a pharmaceutical agent may be mixed into the composition until dissolved or dispersed therein.

In another embodiment, the invention is a method for adhering a polymer film to a water-bearing surface, such as to moistened skin or to a mucosal surface. According to this embodiment of the invention, a composition of any of the embodiments of the invention is provided and applied to the water-bearing surface, thereby causing the composition to adhere to the surface.

In another embodiment, the invention is a method for administering a pharmaceutical agent to an individual in need thereof. According to this embodiment of the invention, a composition of the invention containing therein a pharmaceutical agent is applied to a water-bearing surface, such as moistened skin or a mucosal surface of an individual, thereby causing the composition to adhere to the surface, and causing an effective quantity of the pharmaceutical agent in the composition to be absorbed into the surface.

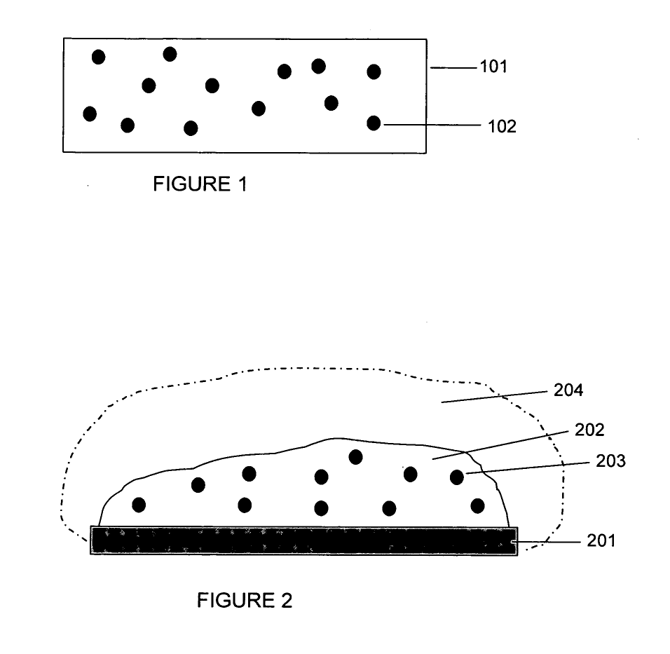

The composition, in all the embodiments of the invention, is a two-phase composition as shown diagrammatically in FIG. 1. The composition includes a finely divided water-soluble polymer 102 which is dispersed in an anhydrous, such as an ethanolic, solvent 101, in which is dissolved a water-insoluble, such as an ethanol-soluble, polymer. This composition, when applied to a water-bearing surface, such as a mucosal surface, will allow for the in situ formation of a semi-solid, mucoadhesive film/membrane that will remain adhered to the surface for extended times.

This composition may be treated, such as by solvent-casting, to produce a dry film. This film may also be applied to a water-bearing surface, such as a mucosal surface, and the film will adhere to this surface.

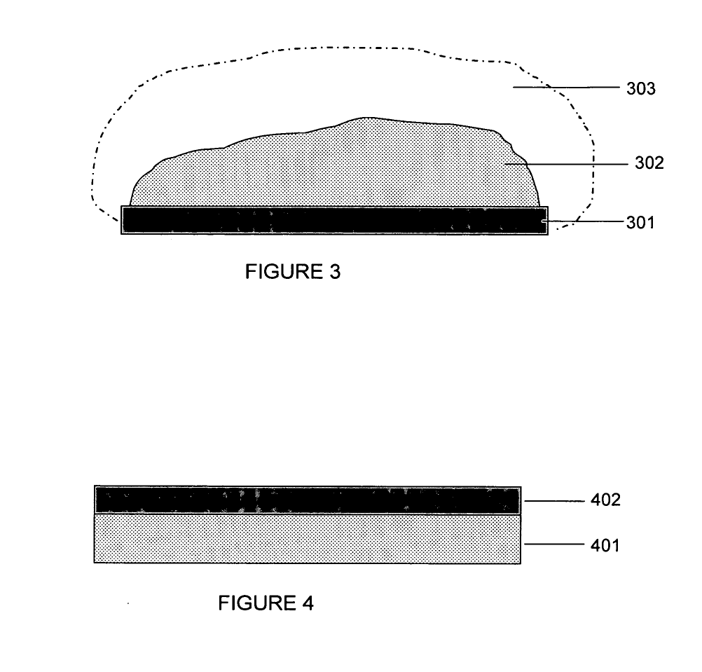

While desiring not to be bound by theory, one reason that the composition of the invention is conceived to be unique is because it contains a bi-phasic (two-phase) nature in relation to an anhydrous solvent, such as an alcoholic solvent like ethanol. Prior art mucoadhesive compositions are soluble in both aqueous and non-aqueous, such as alcoholic, solvents. For example, polyvinylpyrrolidone, polyacrylic acid, and hydroxypropylcellulose are soluble in both an aqueous and an alcoholic solvent. In contrast, the composition of the invention includes an anhydrous solvent-insoluble, such as an ethanol-insoluble, water-soluble mucoadhesive polymer together with a water-insoluble, anhydrous solvent-soluble, such as ethanol-soluble, polymer that provides for the in situ formation of a long-lasting film upon application to a water-bearing, such as a mucosal, surface. In a preferred mode, the water of the water-bearing surface is contained in a mucosal fluid such as saliva. See FIGS. 2 and 3.

FIG. 2 is a diagrammatic representation of a composition of the invention as applied to a water-bearing surface. As shown in FIG. 2, the composition containing a solution 202 of a water-insoluble polymer in a non-aqueous solvent, such as ethanol and a water-soluble polymer 203 that is insoluble in the non-aqueous solvent has been applied to a water-bearing surface 201, such as a mucosal surface, such as a surface within the mouth of a subject, such as on buccal mucosa, resulting in the composition of the invention becoming surrounded by a layer of water 204, such as saliva.

FIG. 3 is a diagrammatic representation of the composition of the invention as shown in FIG. 2 but after remaining adhered to the water-bearing surface for a time sufficient for water from the surface to enter the composition of the invention, causing a swelling of the water-soluble mucoadhesive polymer of the composition, resulting in firm adherence of the composition to the surface. Simultaneously, as water enters the composition, the water-insoluble polymer precipitates, forming a cohesive film/membrane 302 adhered to a water-bearing surface 301 such as mucosa, and surrounded by a layer of water 303, such as saliva.

Because the water-soluble, mucoadhesive polymer is insoluble in the non-aqueous solvent and is present as a dispersed phase, the overall viscosity of the composition remains low and user-friendly prior to application onto a water-bearing surface. However, upon contact with a water-bearing surface, such as moistened skin or a mucosal surface, the composition undergoes a phase transition in which the non-aqueous solvent-soluble, water-insoluble polymer precipitates and forms a cohesive film/membrane. At the same time, the dispersed water-soluble mucoadhesive polymer swells with water, such as in saliva, and adheres to the surface. The combination of both polymers in a suitable, user-friendly solvent system allows for the in situ formation on a water-bearing surface of a film/membrane capable of remaining adhered to the surface for an extended time period.

FIG. 4 shows the mucoadhesive film of the invention 401 coupled with a slowly-dissolving backing layer 402. The backing layer on top of the mucoadhesive film further extends the residence time of the film and, consequently, extends the delivery of a pharmaceutically active agent within the composition, if present.

The non-aqueous solvent that is suitable for the composition of the invention is one, or a multiplicity of solvents, in which a polymer is soluble. Preferably but not necessarily, the non-aqueous solvent is miscible in water. For administration to the body, such as to a mucosal or epidermal surface, it is preferred to use a solvent that is of low toxicity. Examples of non-aqueous solvents include alcohols such as ethanol and benzyl alcohol, propylene glycol, polyethylene glycol, vegetable oils, olive oil, ethyl oleate, and peppermint oil. For application to mucosal surfaces, an alcoholic solvent is preferred and an ethanolic solvent is most preferred. Alcoholic solvents, such as ethanol, may also enhance penetration of a pharmaceutical agent into skin or mucosa.

The water-insoluble polymer that is suitable for the invention will vary depending on the identity of the non-aqueous solvent that is used in the composition. The polymer is soluble in the non-aqueous solvent of the composition and is insoluble in water. Examples of alcoholic solvent-soluble polymers include ethyl cellulose and methacrylate-based polymers, such as those commercially available and marketed under the trade name EUDRAGIT® (Rohm America, Piscataway, N.J.).

The water-soluble polymer of the invention is insoluble in the non-aqueous solvent of the composition of the invention. As defined herein, a polymer is water-soluble if the polymer may be dissolved in water at room temperature at a concentration of 0.1% w/w or higher. Preferably, the polymer may be dissolved in water at a concentration of 1.0% w/w or higher. Polymers that are suitable for the water-soluble polymer of the invention are those polymers that have a bioadhesion index of more than 300. As used herein, the term “bioadhesion index” refers to the ability of a polymer to maintain adherence to a smooth vertical surface that has been wetted with water and is a measure of the residence time of the polymer on the vertical surface prior to dissolution of the polymer in water. The bioadhesion index of a polymer is determined by the test described below in Example 5. It is preferred that the water-soluble polymer of the invention has a bioadhesion index is 500 or more and is most preferred that the bioadhesion index is 800 or more.

In addition, selection of a suitable water-soluble polymer may vary depending on the non-aqueous solvent that is utilized. For example, if the non-aqueous solvent is an alcohol such as ethanol, examples of suitable water-soluble, non-ethanol-soluble polymers are poly(ethylene oxide), polyvinyl alcohol, hydroxypropylmethyl cellulose, guar gum, agar, alginates, pectin, acacia, starch and starch derivatives, polysaccharides, xanthan gum, and gelatin.

The absolute and relative concentrations of the water-soluble polymer and the water-insoluble polymer in the composition of the invention may vary greatly. Typically, the water-soluble polymer concentration in the composition is in the range of 0.4% to 80%. The water-insoluble polymer concentration in the gel formulation is typically in the range of 0.1% to 20%.

The compositions of the invention may optionally contain additional ingredients. For example, the composition may contain a viscosity enhancing agent, such as a polymeric gelling agent that is soluble or dispersible in both water and the non-aqueous solvent of the composition. Examples of such gelling agents include hydroxypropyl cellulose, hydroxypropylmethyl cellulose, polyvinyl alcohol, poly(vinylpyrrolidone) and poly(acrylic acid).

The composition may optionally contain one or more pharmaceutically active drugs that is or are dissolved or dispersed in the non-aqueous solvent of the composition. For example, a drug that may be included in the composition may be an anesthetic, an analgesic, an antibiotic, an anti-inflammatory agent, an antihistamine, an antimicrobial agent such as an antifungal agent, or a hormone such as a steroidal hormone like an estrogen, an androgen, or a progestin. In one embodiment, the composition of the invention contains a corticosteroid, such as hydrocortisone. Other optional ingredients that may be included in the composition of the invention include antimicrobial preservatives, plasticizers such as glycerin or propylene glycol, coloring agents, and flavoring agents such as a mint oil like peppermint or spearmint.

The surface to which the composition of the invention may be applied contains sufficient water for the composition to adhere to the surface, as described above. For example, the surface may be skin that has been moistened, such as by the application of water, the presence of perspiration, or on a wound site that exudes fluid such as blood. The surface may be a mucosal surface, such as the oral mucosa, on which water is present due to the secretion of a bodily fluid such as saliva. Examples of other mucosal surfaces that are suitable for the invention include vaginal mucosa and nasal mucosa. Another example of a surface that is suitable for the invention is tissue at a mucocutaneous junction, such as the lips. The lips may be moistened by licking with the tongue or by the application of water.

When topically applied to the surface, the composition of the invention provides an adherent mucoadhesive film that provides a barrier function. Thus, the invention is useful to protect lesions of the skin or mucosa, such as cuts and scrapes or ulcers. As described above, the composition of the invention may include a pharmaceutically active drug. Adherence of the composition of the invention to a surface provides an extended release of the drug into the surface.

The invention is further illustrated by the following non-limiting examples. In the examples, ethanol is used as an illustration of a non-aqueous solvent. It will be understood that this is merely an example of a non-aqueous solvent that may be used in the invention and that other non-aqueous solvents, such as those listed above, may be used in its place. In the examples, ethylcellulose is used as an illustration of a polymer that is soluble in a non-aqueous solvent and is insoluble in water. It will be understood that this is merely an example of a polymer that is soluble in an alcoholic solvent and that other alcohol-soluble polymers may be used in its place. Moreover, if a non-alcoholic solvent is utilized, a polymer that is soluble in that solvent would be used. In the examples, poly(ethylene)oxide is used as an illustration of a polymer that is soluble in water and insoluble in a non-aqueous solvent such as ethanol. It will be understood that this is merely an example of a polymer that is soluble in an aqueous solvent and insoluble in the non-aqueous solvent of the exemplified composition. Other such polymers may be used in its place and that choice of the water-soluble polymer may be varied, for example, based upon choice of the non-aqueous solvent that is present in the composition.

Additionally, the examples are illustrated with components in addition to the non-aqueous solvent, water-insoluble polymer that is soluble in the non-aqueous solvent, and water-soluble polymer that is insoluble in the non-aqueous solvent. The additional components, such as gelling agent (hydroxypropylcellulose) and pharmaceutically active agent (hydroxycortisone) are optional components and may or may not be present in the compositions of the invention. If such components are present, they may be other than the specific gelling agent and pharmaceutically active agent that are illustrated. Moreover, although not utilized in the examples, other optional ingredients may be included in the composition of the invention.

### EXAMPLE 1

### Mucoadhesive Gel

A mucoadhesive gel formulation was prepared by combining the following components in the following relative concentrations % w/w:

| | |
|---|---|
| Ethanol (200 proof) | 86.6% |
| Ethyl cellulose (EC-N100) | 2.17% |
| Poly(ethylene oxide) (<75 micron) | 8.22% |
| Hydroxypropyl cellulose (HPC GF) | 2.89% |
| Hydrocortisone | 0.13% |

The composition was prepared by the following steps.

1. Poly(ethylene oxide) (Polyox™ 301) (Dow Chemical Company, Midland, Mich.) was sieved using a 200 mesh sieve to produce a particle size of less than 75 micron.

2. Hydrocortisone was added to anhydrous ethanol while stirring at 500 rpm until completely dissolved.

3. Ethyl cellulose was added while stirring at 1000 rpm until fully dissolved.

4. The pre-sieved poly(ethylene oxide) was then added with rapid stirring (1300 rpm).

5. Hydroxypropyl cellulose was then added with rapid stirring (1300 rpm) until completely dissolved.

### EXAMPLE 2

### Solvent-Cast Mucoadhesive Film

A solvent-cast, mucoadhesive film was prepared by casting a thin film of the composition of Example 1 onto a polyester release-liner using a knife over roll batch coater with a gap setting of 1.0 mm. Evaporation of the solvent (ethanol) was accomplished by drying at 60 C for 30 min. The resultant dry film had the following composition:

| | |
|---|---|
| Ethyl cellulose | 16.24% |
| Poly(ethylene oxide) | 61.22% |
| Hydroxypropyl cellulose | 21.48% |
| Hydrocortisone | 1.06% |

### EXAMPLE 3

### Mucoadhesive Film Laminates Including a Backing Layer

The solvent-cast mucoadhesive film of Example 2 was prepared with and without a slowly dissolving backing layer in order to determine the effect of the backing layer on residence time of the mucoadhesive film on a water-bearing surface. After preparing the adhesive film composition of Example 2, a second film was solvent-cast on top of the dry adhesive film and subsequently dried at 60° C. for 30 min. Two-layer laminates prepared in this way included a backing layer made from poly(methacrylic acid—ethyl acrylate) co-polymer family of polymers (Eudragit™ L100 or Eudragit™ S100) (Rohm America, Piscataway, N.J.) or poly(vinyl alcohol). The two-layer laminates are illustrated diagrammatically in FIG. 4.

The laminates were then compared by measuring their in vitro residence times, by attaching discs ( 9/16 inch diameter) of each laminate to a glass slide, followed by immersion in water at room temperature with gentle stirring. The laminates were allowed to remain in water and the residence time was determined. The laminates were also compared by measuring their in vivo residence times, by attaching discs ( 9/16 inch diameter) of each laminate to buccal mucosa in the left buccal pouch of a human subject. Results are shown in Table 1.

TABLE 1

| | | | | |
|---|---|---|---|---|
| | Thickness | | Thickness | Residence |
| Sample | (mm) | Backing | Backing (mm) | Time (min) |

In Vivo Residence Times - Left Buccal Pouch

| | | | | |
|---|---|---|---|---|
| Example 2 | 0.13 | none | n/a | 18 |
| L100 | 0.12 | L100 | 0.01 | 52 |
| S100 | 0.12 | S100 | 0.01 | >106 |
| PVOH | 0.12 | PVOH | 0.01 | 39 |

In Vitro Residence Times - Glass Slide

| | | | | |
|---|---|---|---|---|
| Example 2 | 0.13 | none | n/a | 78 |
| L100 | 0.12 | L100 | 0.01 | 408 |
| S100 | 0.12 | S100 | 0.01 | 155–1053 |
| PVOH | 0.12 | PVOH | 0.01 | 102 |

### EXAMPLE 4

### Mucoadhesive Laminate Adhered to Human Lip

Samples were prepared as described in Example 3, to produce the following two-layer laminate:

### Backing layer=0.010 mm (Eudragit™ S100:Glycerin (9:1)+1.0% Red dye FD&C #40)

### Adhesive layer=0.10 mm (Mucoadhesive Film of Example 2)

Discs of the laminate were punched out ( 7/16 in. diameter) and adhered to the lower lip surface by first moistening the lip prior to application of the disc. The disc was occasionally moistened by the subject's tongue, which allowed disc to hydrate and conform to the topography of the lip surface. Once adhered, the disc became nearly invisible to the naked eye and blended with the adjacent lip color to produce a very aesthetically pleasing appearance. The disc was observed to remain in place, without delamination for about 3 hours. After this time period, the disc began to fragment and was then removed from the lip surface.

### EXAMPLE 5

### Bioadhesion Index

Polymer films were made from poly(vinyl alcohol) (PVOH), sodium carboxymethylcellulose (NaCMC), hydroxypropyl methylcellulose (HPMC), and poly(ethylene oxide) (PEO) by film casting each polymer following dispersal of the polymer in a volatile solvent, such as ethanol. A 7/16 inch circular disc was cut out from each film and was attached to the inner surface of a smooth glass vessel by wetting one side of the disc. The vessel was then filled with water at a temperature of 25° C. to submerge each of the adhered discs and the water was gently stirred using a magnetic stir bar. Visual observation was used to determine residence time of each disc and the mode of termination, whether the disc dissolved or delaminated. Residence times of the polymers were normalized in terms of time in minutes per unit thickness in millimeters to determine the bioadhesion index. The results are shown in Table 2.

TABLE 2

| | THICKNESS | Residence | Bioadhesion | Dissolved or |
|---|---|---|---|---|
| POLYMER | (mm) | Time (min) | Index | Delaminated |
| PVOH | 0.010 | 1 | N.A. | delaminated |
| NaCMC | 0.020 | 6 | 300 | dissolved |
| HPMC | 0.010 | 15 | 1500 | dissolved |
| PEO | 0.050 | 80 | 1600 | dissolved |

As shown in Table 2, hydroxypropyl methyl cellulose and poly(ethylene oxide) have a bioadhesion index higher than 300 and therefore are suitable for the water soluble polymer of the invention. In contrast, sodium carboxymethylcellulose has a bioadhesion index of 300 and is not suitable for the water soluble polymer of the invention. The results of Table 2 are inconclusive regarding poly(vinyl alcohol) because the brief residence time of the film of this polymer was due to delamination from the water-bearing surface rather than dissolution of the polymer. As defined above, bioadhesion index is related to duration of adhesion prior to dissolution and does not pertain to delamination unless the delamination is due to dissolution of polymer.

### EXAMPLE 6

### Additional Mucoadhesive Film Laminates Including a Backing Layer

The following additional mucoadhesive film laminates containing dyclonine HCL and backing layers were made according to the procedures of Examples 2 and 3. The adhesive film was cast at 0.50 mm (wet) and dried at 60° C. for 30 minutes. The backing film layer was cast at 0.10 mm (wet) on top of the adhesive film, then dried at 60° C. for 30 minutes. Circular discs ( 7/16 inch diameter) were punched out from the finished laminate. Each disc contained approximately 3.5 mg of dyclonine HCl.

A. Mucoadhesive Film Laminate 6A

1. Mucoadhesive Film Made According to Example 2

| | % in casting | % in dried |
|---|---|---|
| Component | solution w/w | film w/w |
| Ethanol (200 proof) | 73.19 | 0.00 |
| Glycerin | 2.07 | 7.71 |
| Eudragit (S100) | 0.51 | 1.89 |
| Polyox-301 (<75 micron) | 12.80 | 47.75 |
| Carbopol (C934P) | 2.38 | 8.88 |
| Dyclonine HCl | 9.05 | 33.78 |
| Total Solids | 26.80 | 100.00 |
| Total | 100.00 | 100.00 |

2. Backing Layer

| | % in casting | % in dried |
|---|---|---|
| Component | solution w/w | film w/w |
| Ethanol (200 proof) | 89.95 | 0.00 |
| Glycerin | 2.00 | 19.90 |
| Eudragit (S100) | 8.00 | 79.60 |
| FD&C#40 | 0.050 | 0.495 |
| Total | 100.0 | 100.0 |

B. Mucoadhesive Film Laminate 6B

1. Mucoadhesive Film Made According to Example 2

| | % in casting | % in dried |
|---|---|---|
| Component | solution w/w | film w/w |
| Ethanol (200 proof) | 73.67 | 0.00 |
| Glycerin | 2.01 | 7.63 |
| Ethyl cellulose (EC-N100) | 2.72 | 10.30 |
| Polyox-301 (<75 micron) | 10.25 | 38.91 |
| HPC GF | 2.36 | 8.97 |
| Dyclonine HCl | 9.00 | 34.20 |
| Total Solids | 26.80 | 100.00 |
| Total | 100.00 | 100.00 |

2. Backing Layer

| | % in casting | % in dried |
|---|---|---|
| Component | solution w/w | film w/w |
| Ethanol (200 proof) | 89.92 | 0.00 |
| Glycerin | 2.03 | 20.13 |
| Eudragit (S100) | 8.01 | 79.42 |
| FD&C#40 | 0.05 | 0.49 |
| Total | 100.0 | 100.0 |

Further modifications, uses, and applications of the invention described herein will be apparent to those skilled in the art. It is intended that such modifications be encompassed in the following claims.

1. A mucoadhesive composition comprising two phases, wherein the first phase is an anhydrous solvent in which is dissolved a water-insoluble polymer and the second phase is a water-soluble mucoadhesive polymer that is insoluble in the anhydrous solvent and is dispersed in the anhydrous solvent.

2. The mucoadhesive composition of claim 1 which further contains a pharmacologic agent that is dissolved or dispersed in the anhydrous solvent.

3. The mucoadhesive composition of claim 1 wherein the anhydrous solvent is miscible in water.

4. The mucoadhesive composition of claim 3 wherein the anhydrous solvent is an alcohol.

5. The mucoadhesive composition of claim 4 wherein the alcohol is ethyl alcohol.

6. The mucoadhesive composition of claim 1 wherein the water-soluble polymer has a bioadhesion index greater than 300.

7. The mucoadhesive composition of claim 6 wherein the bioadhesion index is greater than 500.

8. The mucoadhesive composition of claim 6 wherein the bioadhesion index is greater than 800.

9. The mucoadhesive composition of claim 4 wherein the water-soluble polymer is selected from the group consisting of poly(ethylene oxide), polyvinyl alcohol, hydroxypropylmethyl cellulose, guar gum, agar, alginates, pectin, acacia, starch and starch derivatives, polysaccharides, xanthan gum, and gelatin.

10. The mucoadhesive composition of claim 1 wherein the water-insoluble polymer is ethyl cellulose or a methacrylate-based polymer.

11. The mucoadhesive composition of claim 1 that has been applied to a water bearing surface for a time sufficient for water from the surface to enter the composition, and wherein the mucoadhesive polymer is swollen due to the water and the water-insoluble polymer has precipitated, thereby forming a cohesive film.

12. A mucoadhesive film made by solvent-casting the mucoadhesive composition of claim 1.

13. The mucoadhesive film of claim 12 that further comprises a pharmacologic agent dispersed within the film.

14. The mucoadhesive film of claim 12 that has been adhered to a water bearing surface for a time sufficient for water from the surface to enter the film, and wherein the mucoadhesive polymer is swollen due to the water and the water-insoluble polymer has precipitated, thereby forming a cohesive film.

15. A mucoadhesive film laminate comprising, as a first layer, the mucoadhesive film of claim 12 and a second backing layer that adheres to the mucoadhesive film first layer.

16. The mucoadhesive film laminate of claim 15 wherein the backing layer is made by solvent-casting a polymer upon the mucoadhesive film.

17. The mucoadhesive film laminate of claim 15 which further comprises a pharmacologic agent dispersed within the mucoadhesive film.

18. The mucoadhesive film of claim 15 that has been adhered to a water bearing surface for a time sufficient for water from the surface to enter the film, and wherein the mucoadhesive polymer is swollen due to the water and the water-insoluble polymer has precipitated, thereby forming a cohesive film.

19. A method for making a mucoadhesive composition, comprising combining an anhydrous solvent, a water-insoluble polymer that is soluble in the anhydrous solvent, and a water-soluble polymer that is insoluble in the anhydrous solvent, dissolving the water-insoluble polymer in the anhydrous solvent, and dispersing the water-soluble polymer in the anhydrous solvent.

20. The method of claim 19 that further comprises combining a pharmacologic agent and causing the pharmacologic agent to be dissolved or dispersed within the anhydrous solvent.

21. The method of claim 20 that further comprises solvent-casting the mucoadhesive composition, thereby obtaining a mucoadhesive film.

22. The method of claim 21 that further comprises solvent-casting a polymeric backing layer upon the mucoadhesive film obtained by the method of claim 19, thereby obtaining a laminated mucoadhesive film.

23. A method for adhering a polymer film to a water-bearing surface, comprising applying the composition of claim 1 to the water-bearing surface, thereby causing the composition to adhere to the surface.

24. A method for adhering a polymer film to a water-bearing surface, comprising applying the mucoadhesive film of claim 12 to the water-bearing surface, thereby causing the film to adhere to the surface.

25. A method for adhering a polymer film to a water-bearing surface, comprising applying the mucoadhesive film laminate of claim 15 to the water-bearing surface, thereby causing the film laminate to adhere to the surface.

26. A method for administering a pharmaceutical agent to an individual in need thereof, comprising applying the mucoadhesive composition of claim 2 to moistened skin or mucosal surface of an individual in need thereof, thereby causing the composition to adhere to the moistened skin or mucosal surface, and causing an effective quantity of the pharmaceutical agent in the composition to be absorbed into the skin or mucosal surface.

27. A method for administering a pharmaceutical agent to an individual in need thereof, comprising applying the mucoadhesive film of claim 13 to moistened skin or mucosal surface of an individual in need thereof, thereby causing the mucoadhesive film to adhere to the moistened skin or mucosal surface, and causing an effective quantity of the pharmaceutical agent in the film to be absorbed into the skin or mucosal surface.

28. A method for administering a pharmaceutical agent to an individual in need thereof, comprising applying the mucoadhesive film of claim 17 to moistened skin or mucosal surface of an individual in need thereof, thereby causing the mucoadhesive film to adhere to the moistened skin or mucosal surface, and causing an effective quantity of the pharmaceutical agent in the film to be absorbed into the skin or mucosal surface.
